SEQUENCE LISTING (6 sequences)

SEQ ID NO: 1 - DNA, 31 bp, synthetic construct
  source 1..31: mol_type = other DNA
cttgggatatgatgatgaactggtcacctac

SEQ ID NO: 2 - DNA, 30 bp, synthetic construct
  source 1..30: mol_type = other DNA
aagagtagcatcacaatcagaaccttagcc

SEQ ID NO: 3 - DNA, 30 bp, synthetic construct
  source 1..30: mol_type = other DNA
agtacaggactgcaattgctcaatatatcc

SEQ ID NO: 4 - DNA, 30 bp, synthetic construct
  source 1..30: mol_type = other DNA
gcaaagatagcatcacaatcagaaccttag

SEQ ID NO: 5 - DNA, 234 bp, Hepacivirus C
  source 1..234: mol_type = genomic DNA
agtacaggactgcaattgctcaatatatcccggccacgtgacaggtcaccgtatggcttgggatatgatgatgaactggtcacctacagcagccctagtggtatcgcagttactccggatcccacaagctgtcgtggatatggtggcgggggcccattggggagtcctagcgggccttgcctactattccatggtggggaactgggctaaggttctgattgtgatgctactctt

SEQ ID NO: 6 - DNA, 1000 bp, synthetic construct
  source 1..1000: mol_type = other DNA
gagcggtcgcaacctcgtggaaggcgacaacctatccccaaggctcgccagcccgagggtagggcctgggctcagcccgggtacccctggcccctctatggcaatgagggcttggggtgggcaggatggctcctgtcaccccgtggctctcggcctagttggggccccacggacccccggcgtaggtcgcgcaatttgggtaaggtcatcgataccctcacgtgcggcttcgccgatctcatggggtacattccgctcgtcggcgcccccctagggggcgctgccagggccctggcgcatggcgtccgggttctggaggacggcgtgaactatgcaacagggaatctgcccggttgctccttttctatcttccttttggctttgctgtcctgtttgaccatcccagcttccgcttatgaagtgcgcaacgtatccggagtgtaccatgtcacgaacgactgctccaacgcaagcattgtgtatgaggcagcggacatgatcatgcatacccccgggtgcgtgccctgcgttcgggagaacaactcctcccgctgctgggtagcgctcactcccacgctcgcggccaggaacgctagcgtccccactacgacgatacgacgccatgtcgatttgctcgttggggcggctgctctctgctccgctatgtacgtgggagatctctgcggatctgttttcctcgtcgcccagctgttcaccttctcgcctcgccggcacgagacagtacaggactgcaattgctcaatatatcccggccacgtgacaggtcaccgtatggcttgggatatgatgatgaactggtcacctacagcagccctagtggtatcgcagttactccggatcccacaagctgtcgtggatatggtggcgggggcccattggggagtcctagcgggccttgcctactattccatggtggggaactgggctaaggttctgattgtgatgctactctttgccggcgttgacgggggaacctatg

(19) United States
(12) Patent Application Publication
(10) Pub. No.: US 2023/0295752 A1
(43) Pub. Date: Sep. 21, 2023

(54) RAA PRIMERS AND KITS FOR DETECTION OF HEPATITIS C VIRUS

(71) Applicant: ZHENGZHOU UNIVERSITY, Zhengzhou (CN)

(72) Inventors: Aiping WANG, Zhengzhou (CN); Gaiping ZHANG, Zhengzhou (CN); Haili WANG, Zhengzhou (CN); Yuhang ZHANG, Zhengzhou (CN); Yumei CHEN, Zhengzhou (CN); Jingming ZHOU, Zhengzhou (CN); Ying ZHANG, Zhengzhou (CN); Xifang ZHU, Zhengzhou (CN); Chao LIANG, Zhengzhou (CN)

(73) Assignee: ZHENGZHOU UNIVERSITY, Zhengzhou (CN)

(21) Appl. No.: 18/058,851

(22) Filed: Nov. 25, 2022

Related U.S. Application Data
(63) Continuation of application No. PCT/CN2022/126394, filed on Oct. 20, 2022.

(30) Foreign Application Priority Data
Nov. 25, 2021 (CN) 202111415246.1

(51) Int. Cl. C12Q 1/70; G01N 33/543

(52) U.S. Cl. CPC C12Q 1/707; G01N 33/54388; G01N 2333/02

(57) ABSTRACT

The present disclosure disclosed recombinase aided amplification (RAA) primers and kits for the detection of hepatitis c virus. Nucleotide sequences of the RAA primers include: an upstream primer: 5′-FITC-CTTGGGATATGATGATGAACTGGTCACCTAC-3′ (SEQ ID NO. 1); and a downstream primer: 5′-Biotin-AAGAGTAGCATCACAATCAGAACCTTAGCC-3′ (SEQ ID NO. 2). The HCV detection by using the RAA primers screened by the present disclosure has good specificity and high sensitivity (at least 10 copies/μL can be detected). The RAA primers can be used to prepare an HCV detection kit and construct an RAA amplification system. And combined with a lateral flow chromatography technology, the present disclosure can achieve rapid and low-cost detection of HCV and visual result judgment, no complex professional background is required, the use process is convenient and fast, and the detection results are safe and reliable.

### CROSS-REFERENCE TO RELATED APPLICATIONS

This application is a continuation of International Application No. PCT/CN2022/126394, filed on Oct. 20, 2022, which claims priority to Chinese Patent Application No. 202111415246.1, filed on Nov. 25, 2021; the entire contents of which are incorporated herein by reference.

### SEQUENCE LISTING

The instant application contains a Sequence Listing which has been submitted electronically in XML format and is hereby incorporated by reference in its entirety. The XML copy, created on Nov. 24, 2022, is named “2022-11-24-Sequence Listing-62501-0001US00” and is 7,392 bytes in size.

### TECHNICAL FIELD

The present disclosure involves the field of virus biology testing technology, and specifically involves RAA primers and kits for the detection of hepatitis C virus.

### BACKGROUND

Hepatitis C is a disease caused by hepatitis C virus (HCV) and transmitted through blood, and is a class B infectious disease stipulated in the “Prevention and Control of the People's Republic of China”. Science and technology, health and other departments have added hepatitis C-related research into the national science and technology plans to strengthen basic and applied research, support the development of new prevention and treatment technology research, rapid detection technology research and development, and lay the foundation for comprehensively promoting the construction of a healthy China and protecting the health of the people.

At this stage, the conventional diagnostic/detection methods for hepatitis C are qPCR, PCR, and ELISA. Although the three methods are mature, they all require expensive equipment, a long detection period (3-4 hours), and a high cost of reagents and consumables. And at present, they can only be carried out in some municipal Center for Disease Control and Prevention and tertiary medical institutions, and cannot meet the actual needs. Thus, there is an urgent need for a nucleic acid detection method that can be operated in ordinary laboratories, does not require high-cost equipment, and can achieve rapid detection and direct detection result.

The technology developed based on different principles that can make high-efficiency amplification of DNA templates at constant temperatures called the isothermal amplification. In recent years, nucleic acid isothermal amplification has developed rapidly, which can rapidly carry out the amplification reaction in a short time without the limitation of the traditional temperature cycler. Among them, the recombinase polymerase amplification or recombinase aided amplification (RPA/RAA) technology has its own advantages in many aspects including completing the amplification of DNA within 30 min under the constant temperature of 37-42° C., rapid response, simple operation, high specificity and sensitivity, and low requirements for hardware equipment. Thus, RPA/RAA technology is widely used in the disease diagnosis and prevention, especially suitable for in vitro diagnosis, veterinary, food safety, biosecurity, agriculture, and other fields.

The amplification primers of RPA/RAA are the key to the whole reaction, but there is no software or mature design principles for primer design, no data to provide a basis for its primer design, and no mature experience to follow. Therefore, it is imperative to find specific RPA/RAA primers suitable for the hepatitis C virus.

The information disclosed in this background is only for enhancement of understanding of the general background of the present disclosure and should not be taken as an acknowledgment or any form of suggestion that this information forms the prior art already known to a person skilled in the art.

### SUMMARY

The purpose of the present disclosure is to provide a RAA primer and kit for detecting hepatitis C virus, to solve the technical problem that the prior art is difficult to detect HCV rapidly. Combined with a lateral chromatography technology, the RAA primer and kit can solve the problems of the high cost of HCV detection and the difficulty of quickly and intuitively presenting HCV detection results.

In order to solve the above technical problems, the present disclosure uses the following technical solutions.

Recombinase aided amplification (RAA) primers for detecting hepatitis C virus is obtained by screening, and nucleotide sequences of the RAA primers is as follows:

an upstream primer:
(SEQ ID NO. 1)
5′-FITC-CTTGGGATATGATGATGAACTGGTCACCTAC-3′;
a downstream primer:
(SEQ ID NO. 2)
5′-Biotin-AAGAGTAGCATCACAATCAGAACCTTAGCC-3′.

In some embodiments, the following nucleotide sequences are also included in the RAA primers:

an upstream primer:
(SEQ ID NO. 3)
5′-FITC-AGTACAGGACTGCAATTGCTCAATATATCC-3′;
a downstream primer:
(SEQ ID NO. 4)
5′-Biotin-GCAAAGATAGCATCACAATCAGAACCTTAG-3′.

The RAA primers can be applied to the preparation of products related to the detection of hepatitis C virus, such as an HCV detection kit, an RAA amplification system:

The HCV detection kit includes the RAA primers, as well as an RNA extraction reagent, an RAA amplification reaction reagent, a lateral flow chromatography test strip, reaction dry powder, and a positive control sample, etc. In some embodiments, the HCV detection kit further includes a negative control sample. The RNA extraction reagent may be a reagent used to extract RNA from a sample to be detected. In some embodiments, the RNA extraction reagent may be, for example, a Trizol-RNA reagent or reagents of an RNA extraction kit (e.g., QIAamp Viral RNA Mini Kits). The RAA amplification reaction reagent is a reagent including a buffer and a magnesium acetate solution, the buffer including 200 mmol/L of HEPES at pH 6.8, 600 mmol/L of NaCl solution, and 60 mmol/L of MgCl₂ solution. The positive control sample is a plasmid including C/E1 gene of the hepatitis C virus.

The RAA amplification system for HCV detection includes:

41.5 μL of a buffer, 10 μL of deionized water, 2.5 μL of each of the upstream primer and downstream primer, 2 μL of an RNA extract of a sample to be tested, reaction dry powder, and 2.5 μL of 280 mmol/L magnesium acetate solution. The buffer includes 200 mmol/L of HEPES at pH 6.8, 600 mmol/L of NaCl solution, and 60 mmol/L of MgCl₂ solution. The reaction dry powder is a cre recombinase at 50 μg/ml. The sample to be test may include peripheral blood, serum, etc., of a subject (e.g., a patient).

A method of using the HCV detection kit includes the following steps:
(1) extracting the RNA from the sample to be tested;
(2) obtaining a positive plasmid pUC57-pC/E1 as a positive standard;
(3) using a recombinase aided amplification and lateral flow assay (RAA-LFA) to detect the hepatitis C virus.

Specifically, using an RNA extraction reagent to extract RNA from a sample to be tested, and obtaining a RAA amplification product by performing an isothermal amplification reaction on the RNA extract by using the RAA primers and the RAA amplification reaction reagent.

The isothermal amplification reaction includes: adding 41.5 μL of buffer, 10 μL of deionized water, 2.5 μL of each of the upstream primer and downstream primer, 2 μL of each of the RNA extract or the positive plasmid to a 0.2 mL detection tube containing reaction dry powder, and finally adding 2.5 μL of 280 mmol/L magnesium acetate solution for thoroughly mixing to obtain a mixed RAA amplification system, placing the mixed RAA amplification system on a water bath, and reacting at 30-42° C. for 5-30 min to obtain the RAA amplification product. The buffer includes 200 mmol/L of HEPES at pH 6.8, 600 mmol/L of NaCl solution, and 60 mmol/L of MgCl₂ solution, the reaction dry powder is a cre recombinase at 50 μg/ml.

In some embodiments, the positive control sample is a positive control using positive plasmid pUC57-pC/E1 as template, which includes HCV genome fragment, e.g., C/E1 gene). In some embodiments, a negative control sample (negative control using empty vector plasmid without HCV genome fragment compared with the positive plasmid pUC57-pC/E1) is used in the method. The sequence of the plasmid pUC57-pC/E1 is shown in SEQ ID NO. 6

(GAGCGGTCGCAACCTCGTGGAAGGCGACAACCTAT
CCCCAAGGCTCGCCAGCCCGAGGGTAGGGCCTGGGC
TCAGCCCGGGTACCCCTGGCCCCTCTATGGCAATGA
GGGCTTGGGGTGGGCAGGATGGCTCCTGTCACCCCG
TGGCTCTCGGCCTAGTTGGGGCCCCACGGACCCCCG
GCGTAGGTCGCGCAATTTGGGTAAGGTCATCGATAC
CCTCACGTGCGGCTTCGCCGATCTCATGGGGTACAT
TCCGCTCGTCGGCGCCCCCCTAGGGGGCGCTGCCAG
GGCCCTGGCGCATGGCGTCCGGGTTCTGGAGGACGG
CGTGAACTATGCAACAGGGAATCTGCCCGGTTGCTC
CTTTTCTATCTTCCTTTTGGCTTTGCTGTCCTGTTT
GACCATCCCAGCTTCCGCTTATGAAGTGCGCAACGT
ATCCGGAGTGTACCATGTCACGAACGACTGCTCCAA
CGCAAGCATTGTGTATGAGGCAGCGGACATGATCAT
GCATACCCCCGGGTGCGTGCCCTGCGTTCGGGAGAA
CAACTCCTCCCGCTGCTGGGTAGCGCTCACTCCCAC
GCTCGCGGCCAGGAACGCTAGCGTCCCCACTACGAC
GATACGACGCCATGTCGATTTGCTCGTTGGGGCGGC
TGCTCTCTGCTCCGCTATGTACGTGGGAGATCTCTG
CGGATCTGTTTTCCTCGTCGCCCAGCTGTTCACCTT
CTCGCCTCGCCGGCACGAGACAGTACAGGACTGCAA
TTGCTCAATATATCCCGGCCACGTGACAGGTCACCG
TATGGCTTGGGATATGATGATGAACTGGTCACCTAC
AGCAGCCCTAGTGGTATCGCAGTTACTCCGGATCCC
ACAAGCTGTCGTGGATATGGTGGCGGGGGCCCATTG
GGGAGTCCTAGCGGGCCTTGCCTACTATTCCATGGT
GGGGAACTGGGCTAAGGTTCTGATTGTGATGCTACT
CTTTGCCGGCGTTGACGGGGGAACCTATG).

Further, the method further includes dripping the obtained RAA amplification product to a sample well of a colloidal gold-labeled lateral flow chromatography test strip, and after reaction for 3-10 min, determining a detection result by observing the appearance of a detection line and/or a control line on the colloidal gold-labeled lateral flow chromatography test strip.

The method further includes first incubating the RAA amplification product at 37° C. for 5 min, then diluting the RAA amplification product with 200 μL of a PBST buffer, dripping the diluted RAA amplification product to the sample well of the colloidal gold-labeled lateral flow chromatography test strip, and after reaction at room temperature for 5-10 min, determining the detection result by observing the appearance of the detection line and/or the control line on the colloidal gold-labeled lateral flow chromatography test. The detection line is red, indicating that the sample to be test is positive (Positive Readout), and the detection line is not colored, indicating that the sample to be test is negative (Negative Readout).

Compared with the prior art, the main beneficial technical effects of the present disclosure are as follows.
1. The present disclosure comprehensively considers many factors such as target region situations, lengths of the RAA primers and possibilities of forming secondary structure, GC contents, lengths of the amplified product, etc. The RAA primers that can be used for the rapid detection of HCV were obtained by screening, with good specificity and high sensitivity (at least 10 copies/μL can be detected).
2. The present disclosure combines RPA/RAA with Lateral Flow Assay (LFA), and thus, a user without a complex professional background can quickly realize a visual judgment of HCV detection results.
3. The HCV detection using the kit of the present disclosure mainly includes two steps: isothermal amplification and test strip result determination. The amplification step takes 10 minutes, and the result determination step takes 3 to 5 minutes. Compared with the traditional RT-PCR method, nearly 3 hours can be shortened, which can meet the needs of rapid and high-throughput screening on-site. In addition, the detection process is convenient and fast, and the detection results are safe and reliable.

### BRIEF DESCRIPTION OF THE DRAWINGS

FIG. 1 is an exemplary graph showing the results of a sensitivity experiment based on the RAA-LFA detection method according to some embodiments of the present disclosure.

FIG. 2 is an exemplary graph showing the results of a specificity experiment based on the RAA-LFA detection method according to some embodiments of the present disclosure.

### DETAILED DESCRIPTION

The specific embodiments of the present disclosure will be described below in conjunction with the accompanying drawings and embodiments, but the following embodiments are only used to describe the present disclosure in detail and do not limit the scope of the present disclosure in any way.

Additionally, for numerical ranges in the present disclosure, it should be understood that each intervening value between the upper and lower limits of the range is also specifically disclosed. Every smaller range between any stated value or intervening value in a stated range and any other stated value or intervening value in that stated range is also encompassed within the present disclosure. The upper and lower limits of these smaller ranges may independently be included or excluded in the range.

Unless otherwise defined, all technical and scientific terms used in the present disclosure have the same meaning as commonly understood by one of ordinary skill in the art to which the present disclosure relates. Although only the preferred methods and materials are described herein, any methods and materials similar or equivalent to those described in the present disclosure can also be used in the practice or testing of the present disclosure. All documents mentioned in the present disclosure are incorporated by reference for the purpose of disclosing and describing the methods and/or materials in connection with which the documents are referred. In the event of a conflict with any incorporated document, the content of the present disclosure controls.

### Embodiment 1: Primer Design and Condition Optimization

The key to RAA amplification lies in the design of primers, but RAA is different from conventional PCR reactions. At present, there is no software or mature design principles for RAA primer design, and there is no large amount of data to provide a basis for its primer design. There is no mature experience to follow. Based on long-term practical research, the present disclosure selects the C/E1 genes of the HCV 1b genotype strain and other 6 genotype strains included in the NCBI for sequence comparison, and prudently selects the conserved region as the target of RAA amplification. The nucleotide sequence of the conserved region of C/E1 gene is shown in SEQ ID NO. 5 (AGTACAGGACTGCAATTGCTCAATATATCCCGGCCACGTGACAGGTCACCGTATG GCTTGGGATATGATGATGAACTGGTCACCTACAGCAGCCCTAGTGGTATCGCAGTT ACTCCGGATCCCACAAGCTGTCGTGGATATGGTGGCGGGGGCCCATTGGGGAGT CCTAGCGGGCCTTGCCTACTATTCCATGGTGGGGAACTGGGCTAAGGTTCTGATT GTGATGCTACTCTT). A plurality of factors has been comprehensively considered, such as 1. GC content (40-60%); 2. length (30-35nt); 3. the size of the amplification product (100-300 bp) and so on. Primer design includes upstream primer design and downstream primer design. According to the designed upstream RAA primers and downstream RAA primers, cross-pairing was carried out, and paired primers were screened to obtain the optimal primers. See Table 1 for details.

TABLE 1
designed RAA primers for the conserved
region of the C/E1 gene of
hepatitis C virus

| | SEQ | | |
|---|---|---|---|
| Primer | ID | | Location |
| name | NO | Sequence (5′-3′) | (nt) |
| RAA-F1 | 3 | AGTACAGGACTGCAA | 1241-1270 |
| | | TTGCTCAATATATCC | |
| RAA-R1 | 4 | GCAAAGATAGCATCA | 1477-1447 |
| | | CAATCAGAACCTTAG | |
| RAA-F2 | 1 | CTTGGGATATGATGA | 1297-1327 |
| | | TGAACTGGTCACCTA | |
| | | C | |
| RAA-R2 | 2 | AAGAGTAGCATCACA | 1474-1445 |
| | | ATCAGAACCTTAGCC | |

The specificity and amplification efficiency were further evaluated, and a more preferred primer pair was screened, specifically: after RAA amplification was performed with the RAA primer pairs designed in Table 1, agarose gel electrophoresis and lateral chromatography technology were performed. The F2/R2 primer pair after being screened has best amplification effect, good specificity and no non-specific amplification. However, other primer pairs have problems such as non-specific amplification and low amplification efficiency. Therefore, the F2/R2 primer pair was selected for the subsequent optimization of RAA reaction conditions, as well as the specificity and sensitivity test. The specific nucleotide sequence of the primer pair F2/R2 is: an upstream primer: 5′-FITC-CTTGGGATATGATGATGAACTGGTCACCTAC-3′ (SEQ ID NO.1); a downstream primer: 5′-Biotin-AAGAGTAGCATCACAATCAGAACCTTAGCC-3′ (SEQ ID NO. 2).

The RAA reaction time and reaction temperature were optimized, and it was found that the RAA amplification efficiency was the highest when the reaction temperature was 37° C. and the reaction was performed for 5 min. The optimized RAA reaction conditions of the present disclosure require only one temperature for the entire gene amplification process, do not require special instruments, are simpler to operate, and are suitable for on-site rapid detection of the hepatitis C virus.

### Embodiment 2: Sensitivity and Specificity Test

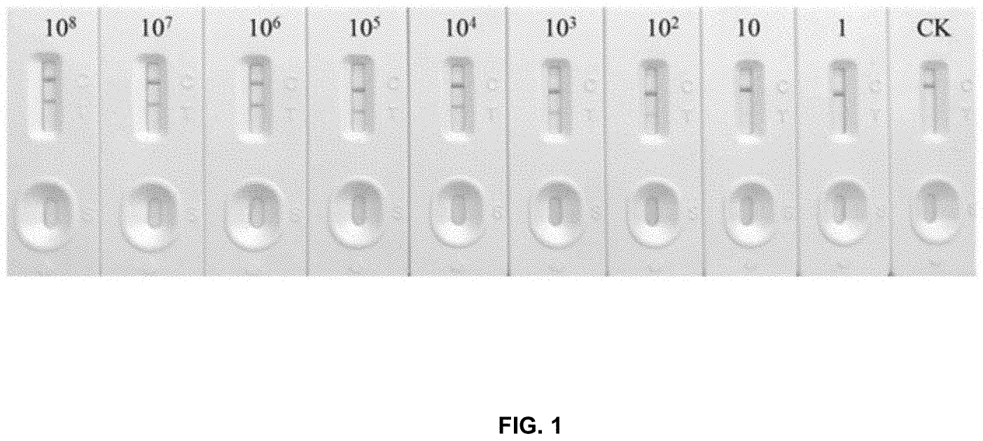

A standard hepatitis C virus plasmid sample was selected as a template, and the template was diluted 10-fold gradient with double distilled water to a standard plasm id template with a concentration of 108 copies/μL-1copy/μL. Double distilled water was used as a blank control (CK). The optimized reaction conditions and the primer pair F2/R2 described in embodiment 1 were used for the RAA detection. The result is that the minimum detected template amount is 10 copies/μL (see FIG. 1). The sensitivity is higher than that of the conventional PCR method. The detection method of the present disclosure is simpler and faster, especially suitable for on-site detection, and has looser requirements on experimental conditions (e.g., small concentration of samples can be detected).

The nucleic acid samples were extracted from the positive samples of hepatitis A virus (HAV), hepatitis B virus (HBV), human immunodeficiency virus (HIV), Syphilis, and Human Papillomavirus (HPV) among the six infectious diseases as templates, and RAA was carried out according to the optimized reaction conditions and primer pair F2/R2 in Embodiment 1, and detection was performed by gel electrophoresis and lateral chromatography technology.

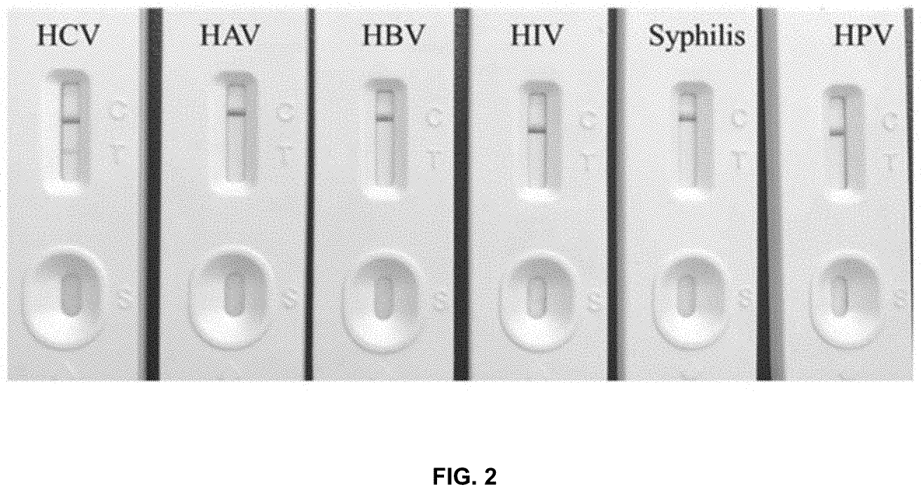

The experimental results of the lateral chromatography technology are shown in FIG. 2, only the hepatitis C virus (HCV) is positive, and the others are all negative, indicating that the RAA detection system of the present disclosure has good specificity.

### Embodiment 3: Validation Test Applied to Individual Patient Screening

2 mL of peripheral blood in vitro of a patient were taken and centrifuged at 3000 rpm for 5 min, and 200 μL of supernatant were taken to add into 560 μL of AVL lysate containing 5.6 μg of Carrier RNA. Viral RNA was extracted according to the instruction of QIAamp Viral RNA Mini Handbook (Qiagen, catalog #52904/52906), elution volume is 50 μL. According to the instruction of the RAA kit, 41.5 μL of buffer (which includes 200 mmol/L of HEPES at pH 6.8, 600 mmol/L of NaCl solution, and 60 mmol/L of MgCl₂ solution), 10 μL of deionized water, 2.5 μL of each upstream primer and downstream primer of the primer pair F2/R2, 2 μL of RNA extract from the patient (2 μL of a positive template plasmid is added to another detection tube, the other condition is the same as the RNA extract), and finally 2.5 μL of 280 mmol/L magnesium acetate solution were added to a 0.2 mL detection tube containing reaction dry powder (The reaction dry powder cre recombinase at 50 μg/ml. The cre recombinase is used to promote RAA amplification, for example, for RNA extract, the cre recombinase may convert RNA to DNA for amplification; for DNA extract, the cre recombinase may promote DNA amplification directly) to obtain a RAA amplification system, the RAA amplification system was thoroughly mixed, the mixed RAA amplification system was placed on a water bath for reaction at 30-42° C. for 5-30 min to obtain the RAA amplification product. The amplification product was directly added dropwise to the colloidal gold-labeled lateral flow chromatography test strip, and the resulting bands were clear. For a positive sample, the detection line (T line) was red which could be distinguished by the naked eye, and for a negative sample, the detection line (T line) was not colored. The control line (line C) indicates that the test result is valid.

### Embodiment 4: Validation Test Applied to Multiple Patients

2 mL of peripheral blood from each patient was taken and centrifuged at 3000 rpm for 5 min, and 200 μL of supernatant were taken. The viral RNA of each serum was extracted by using an automatic nucleic acid extractor, with an elution volume of 50 μL. According to the instruction of RAA kit, 41.5 μL of buffer, 10 μL of deionized water, 2.5 μL of each upstream primer and downstream primer, 2 μL of RNA extract from each patient (2 μL of a positive template plasmid is added to another detection tube, the other condition is the same as the RNA extract), and finally 2.5 μL of 280 mmol/L magnesium acetate solution were added to a 0.2 mL detection tube containing reaction dry powder to obtain a RAA amplification system, the RAA amplification system was thoroughly mixed, the mixed RAA amplification system was placed on a water bath for reaction at 30-42° C. for 5-30 min to obtain the RAA amplification product. The amplification product was directly added dropwise to the colloidal gold-labeled lateral flow chromatography test strip, and the resulting band was clear. For a positive sample, the detection line (T line) was red which could be distinguished by the naked eye, and for a negative sample, the detection line (T line) was not colored. The control line (line C) indicates that the test result is valid.

The present disclosure has been described in detail above in conjunction with the accompanying drawings and embodiments. However, those skilled in the art can understand that changes, modifications, substitutions, combinations, and simplifications made without departing from the concept of the present disclosure should be equivalent alternatives, thereby forming multiple specific embodiments, that are within the scope of common variations of the present disclosure.

What is claimed is:

1. Recombinase aided amplification (RAA) primers for detecting hepatitis C virus, wherein nucleotide sequences of the RAA primers comprise:
an upstream primer:
(SEQ ID NO. 1)
5′-FITC-CTTGGGATATGATGATGAACTGGTCACCTAC-3′;
and
a downstream primer:
(SEQ ID NO. 2)
5′-Biotin-AAGAGTAGCATCACAATCAGAACCTTAGCC-3′.

2. The RAA primers according to claim 1, wherein nucleotide sequences of the RAA primers further comprise:
an upstream primer:
(SEQ ID NO. 3)
5′-FITC-AGTACAGGACTGCAATTGCTCAATATATCC-3′;
and
a downstream primer:
(SEQ ID NO. 4)
5′-Biotin-GCAAAGATAGCATCACAATCAGAACCTTAG-3′.

3. A hepatitis C virus detection kit, wherein the hepatitis C virus detection kit comprises the recombinase aided amplification (RAA) primers of claim 1.

4. The hepatitis C virus detection kit according to claim 3, further comprising an RNA extraction reagent, an RAA amplification reaction reagent, a lateral flow chromatography test strip, reaction dry powder, and a positive control sample.

5. The hepatitis C virus detection kit according to claim 4, wherein the RAA amplification reaction reagent is a reagent including a buffer and a magnesium acetate solution, the buffer including 200 mmol/L of HEPES at pH 6.8, 600 mmol/L of NaCl solution, and 60 mmol/L of MgCl₂ solution.

6. The hepatitis C virus detection kit according to claim 4, wherein the positive control sample is a plasmid including C/E1 gene of the hepatitis C virus.

7. A recombinase aided amplification (RAA) amplification system for detecting hepatitis C virus, wherein the RAA amplification system comprises:
41.5 μL of a buffer, 10 μL of deionized water, 2.5 μL of each of the upstream primer and downstream primer according to claim 1, 2 μL of an RNA extract of a sample to be tested, reaction dry powder, and 2.5 μL of a magnesium acetate solution in 280 mmol/L, wherein the buffer includes 200 mmol/L of HEPES at pH 6.8, 600 mmol/L of NaCl solution, and 60 mmol/L of MgCl₂ solution, the reaction dry powder is a cre recombinase at 50 μg/ml.

8. A method of using the hepatitis C virus detection kit of claim 4, wherein the method comprises the following steps:
using the RNA extraction reagent to extract RNA from a sample to be tested, and obtaining an RAA amplification product by performing an isothermal amplification reaction on the RNA extract.

9. The method according to claim 8, wherein the isothermal amplification reaction includes:
adding 41.5 μL of a buffer, 10 μL of deionized water, 2.5 μL of each of the upstream primer and downstream primer, 2 μL of each of the RNA extract or the positive control sample to a 0.2 mL detection tube containing reaction dry powder, wherein the buffer includes 200 mmol/L of HEPES at pH 6.8, 600 mmol/L of NaCl solution, and 60 mmol/L of MgCl₂ solution, the reaction dry powder is a cre recombinase at 50 μg/ml, and finally adding 2.5 μL of 280 mmol/L magnesium acetate solution for thoroughly mixing to obtain a mixed RAA amplification system,
placing the mixed RAA amplification system on a water bath, and
reacting at 30-42° C. for 5-30 min to obtain the RAA amplification product.

10. The method according to claim 9, wherein the method further comprises the following steps: dripping the obtained RAA amplification product to a sample well of a colloidal gold-labeled lateral flow chromatography test strip, and after reaction for 3-10 min, determining a detection result by observing the appearance of a detection line and/or a control line on the colloidal gold-labeled lateral flow chromatography test strip.

11. The method according to claim 10, wherein the method further comprises: first incubating the RAA amplification product at 37° C. for 5 min, diluting the RAA amplification product with 200 μL of a PBST buffer, dripping the diluted RAA amplification product to the sample well of the colloidal gold-labeled lateral flow chromatography test strip, and after reaction at room temperature for 5-10 min, determining the detection result by observing the appearance of the detection line and/or the control line on the colloidal gold-labeled lateral flow chromatography test strip.
